(19) United States
(12) Patent Application Publication
(10) Pub. No.: US 2004/0093019 A1
(43) Pub. Date: May 13, 2004

(54) Surgical instrument

(76) Inventor: Lutz Kothe, Radolfzell (DE)

Correspondence Address: BACHMAN & LAPOINTE, P.C., 900 CHAPEL STREET, SUITE 1201, NEW HAVEN, CT, 06510, US

(21) Appl. No.: 10/688,153

(22) Filed: Oct. 17, 2003

(30) Foreign Application Priority Data
Nov. 5, 2002 (DE) 102 51 785.1

(51) Int. Cl. A61B 17/28

(52) U.S. Cl. 606/205

(57) ABSTRACT

In a surgical instrument with a pulling or pushing element for actuating at least one jaw part, the pulling or pushing element is to be assigned at least one bar.

### (1) FIELD OF THE INVENTION

The invention relates to a surgical instrument with a pulling or pushing element for actuating at least one jaw part.

### (2) DESCRIPTION OF THE RELATED ART

The present case involves in particular surgical instruments with tubular shafts used in endoscopy. These instruments are used, for example, to perform various surgical procedures within the human body. They are mainly distinguished by the fact that a pulling or pushing element for the working elements, in particular for jaw parts, is arranged displaceably in a protective tube, said jaw parts being located at one end of the protective tube, while the pulling or pushing element is connected at the other end to grip arms with which the pulling or pushing element and thus the jaw parts are moved. Reference is made purely by way of example to WO 95/15720 or DE 43 41 735 C1.

In view of the hygiene demands placed on surgical instruments nowadays, it is imperative that the instrument be cleaned thoroughly after the surgical intervention. This is highly problematic in the case of surgical instruments with a closed protective tube and with a pulling or pushing element lying inside the latter, because these instruments have to be dismantled and flushed clean. In practice, this cleaning procedure can be monitored only in a very unsatisfactory way.

### SUMMARY OF THE INVENTION

The object of the present invention is to develop a surgical instrument of the above-mentioned type which can be cleaned much better. In addition, it should also be possible for the cleaning to be better monitored.

This object is achieved by the fact that the pulling or pushing element is assigned at least one bar.

This bar (note however that two bars are preferably used) serves to stabilize the guiding of the pulling or pushing element and preferably also to connect it to at least one jaw part. This means, however, that the protective tube now only has a protective function, and no longer any actual working function, so that it can have a much simpler design. For this reason, it is also possible for the protective tube to be made, for example, as a plastic disposable article, so that it no longer has to be cleaned. To clean the surgical instrument, the protective tube is removed from the bars and the pulling or pushing element and discarded, and, after the bars and the pulling or pushing element have been cleaned, is replaced by a new one.

After removal of the protective tube, however, all parts of the bars and of the pulling or pushing element are exposed and can thus be very easily reached by suitable cleaning agents. A visual check of these reusable parts is also easily possible.

Where two bars are provided, these are preferably connected to one another. The connection can be made via suitable connecting bridges, clamping rings, guide rivets or the like. If desired, it is of course also possible for a plurality of bars to be provided.

To ensure that the space between the pulling or pushing element and the bars can be easily cleaned, both are supported against one another via spacers. These spacers can be formed integrally on the pulling or pushing element or else also on the bars.

The bars preferably have an outer contour which matches an inner surface of the tube. This makes it easier to push the protective tube on.

In this embodiment it is now possible to connect the ends of the corresponding jaw parts to the bars. This connection is preferably hinged, so that the jaw parts can be pivoted relative to the bars.

In one illustrative embodiment, the ends of the bars each have a bead which engages in a groove in the jaw part, said groove turning partially about the bead. In this case it may prove advisable to make the end of the bar before the bead elastic, so that the bars can spread apart at the ends. In this way, upon closure of the jaw parts, a closing pressure is also exerted on the jaw parts so that the work of the operating surgeon, for example cutting of tissue, is made easier.

The ends of the two jaw parts are also connected to one another via a hinge pin and connected to the pulling or pushing element, so that the jaw opens or closes as the pulling or pushing element is moved.

In another illustrative embodiment of the invention for which protection is also sought separately, the pulling or pushing element engages with a bead in a groove in the jaw part. The jaw part in turn sits with a rotating end in a recess in the other jaw part and rotates with this end in this recess. To ensure that the movement of the jaw part in this recess can be controlled, the rotating end of the movable jaw part in the recess has an abutment tip which bears against an inner wall of the recess. This abutment tip forms a fixed point of rotation.

This design of the jaw mechanics has the considerable advantage that no bores and no riveted joints are needed to permit opening or closing of the jaw. By means of this design, weak points caused by bore holes or by breaking of rivets are eliminated.

### BRIEF DESCRIPTION OF THE DRAWINGS

Further advantages, features and details of the invention will become clear from the following description of preferred illustrative embodiments and with reference to the drawing, in which

FIG. 1 shows a perspective view of a detail of a surgical instrument according to the invention;

FIG. 2 shows a front view of the detail of the surgical instrument according to FIG. 1;

FIG. 3 shows a front view of a detail, corresponding to FIG. 1, of another illustrative embodiment of a surgical instrument;

FIG. 4 shows a perspective view of a detail of a further illustrative embodiment of a surgical instrument;

FIG. 5 shows a front view of a detail of a further illustrative embodiment of a surgical instrument;

FIG. 6 shows a plan view of parts of a surgical instrument, with jaw parts in the open position;

FIG. 7 shows a plan view of parts of the surgical instrument corresponding to FIG. 6, with jaw parts in the closed position;

FIG. 8 shows a side view of a jaw part according to the invention;

FIG. 9 shows a plan view of the jaw part according to FIG. 8;

FIG. 10 shows a side view of a front end of a bar according to the invention;

FIG. 11 shows a side view of a further illustrative embodiment of a jaw part;

FIG. 12 shows a side view of the front end of an attachment piece according to the invention with rigid jaw part;

FIG. 13 shows a side view of an element from the front area of a further illustrative embodiment of a surgical instrument, with a jaw part in the open position;

FIG. 14 shows a side view of the front area of a surgical instrument according to FIG. 13, with the jaw part in the closed position.

### DETAILED DESCRIPTION OF PREFERRED EMBODIMENTS

The surgical instrument according to the present invention is in principle a tubular shaft instrument, such as is shown in WO 95/15720 for example. In the latter, working elements, in particular jaw parts, are moved for the purpose of performing an intervention in a human body for example. The movement of the jaw parts is effected via a pulling or pushing element which is connected to at least one grip arm of a handpiece.

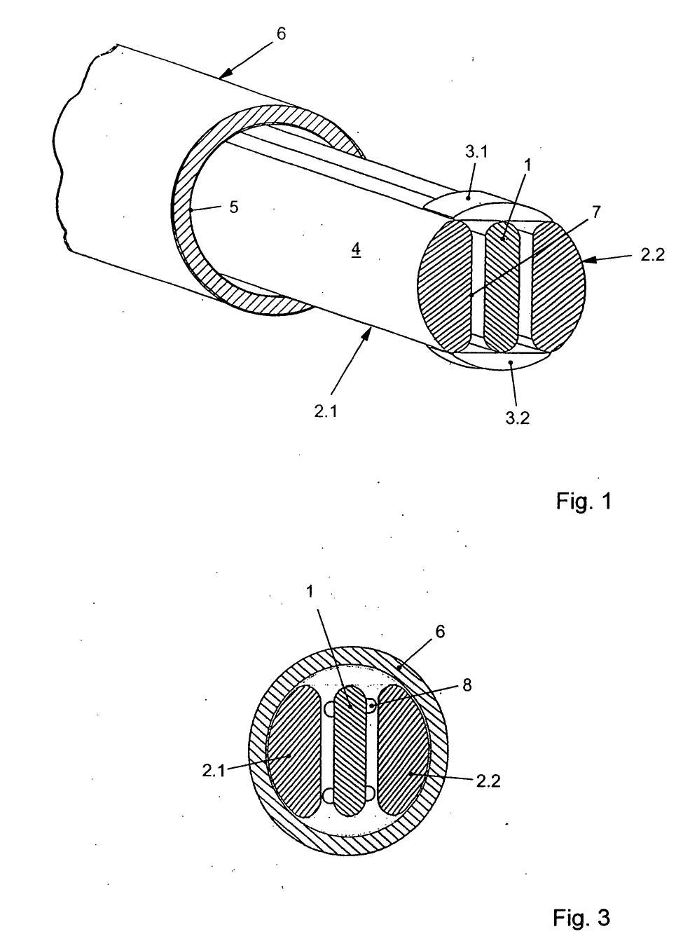

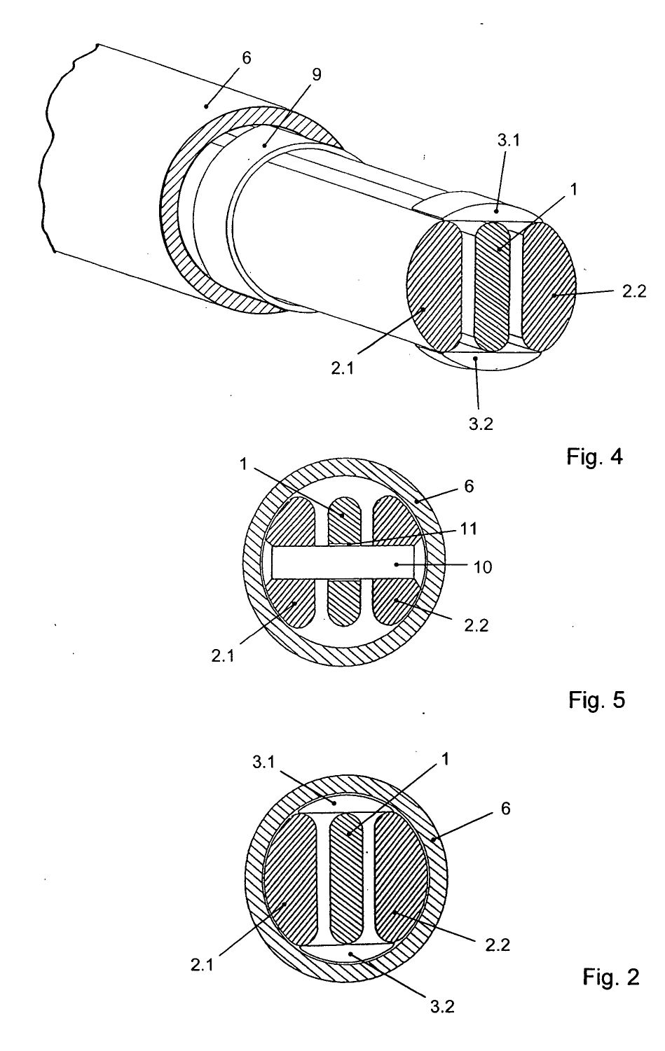

In FIG. 1 this pulling or pushing element is indicated by 1 and is designed as a flat rail. According to the invention, the pulling or pushing element moves between two bars 2.1 and 2.2, said bars 2.1 and 2.2 being connected to one another via connecting bridges 3.1 and 3.2. A respective outer contour 4 of the bars 2.1 and 2.2 has a rounded configuration and matches an inner surface 5 of a protective tube 6. The connecting bridges 3.1 and 3.2 also have a rounded configuration.

By contrast, an inner contour 7 of the bars 2.1 and 2.2 has a flat configuration, so that the pulling or pushing element can slide along it. Spacers 8 can be provided on the bars 2.1 and 2.2 or also on the pulling or pushing element 1, as is shown in FIG. 3.

The illustrative embodiment of a surgical instrument according to FIG. 4 differs from that in FIG. 1 in that the unit made up of the two bars 2.1 and 2.2 and of the pulling or pushing element 1 is held together by a clamping ring 9.

According to the illustrative embodiment of a surgical instrument according to FIG. 5, the two bars 2.1 and 2.2 are held together by a connecting element 10, which for example can be designed as a guide rivet. This connecting element 10 also extends through the pulling or pushing element 1, a corresponding oblong hole 11 being able to be provided in the area of the connecting element 10 so that the pulling or pushing element 1 retains its freedom of movement.

The way in which the present invention functions is as follows:

In the assembled state, the protective tube 6 encloses the bars 2.1 and 2.2 and their connecting bridges 3.1 and 3.2 and the clamping ring 9. The pulling or pushing element 1 can be pushed to and fro between the bars 2.1 and 2.2 by moving the above-mentioned grip arm, so as to permit opening and closing of corresponding jaw parts.

If the instrument is to be cleaned, the protective tube 6 is removed, and the unit made up of the bars 2.1 and 2.2 and the pulling or pushing element 1 is subjected to a suitable cleaning solution or a cleaning bath. The spacers 8 ensure that the pulling or pushing element is at a distance from the bars 2.1 and 2.2, so that a cleaning solution can flow unrestrictedly about all the parts. The state of cleaning can be easily checked either visually or by other means.

The protective tube 6 is preferably made of plastic so that it can be a disposable article. After the other parts have been cleaned, a new protective tube can be pushed onto the unit made up of bars 2.1 and 2.2 and pulling or pushing element 1 from the direction of the jaw part or from the direction of the handgrip.

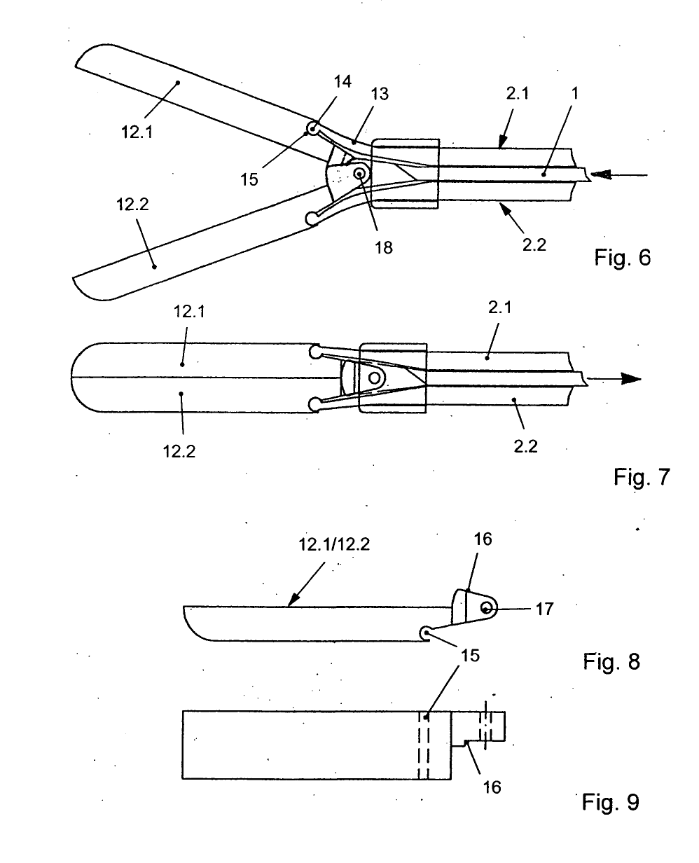

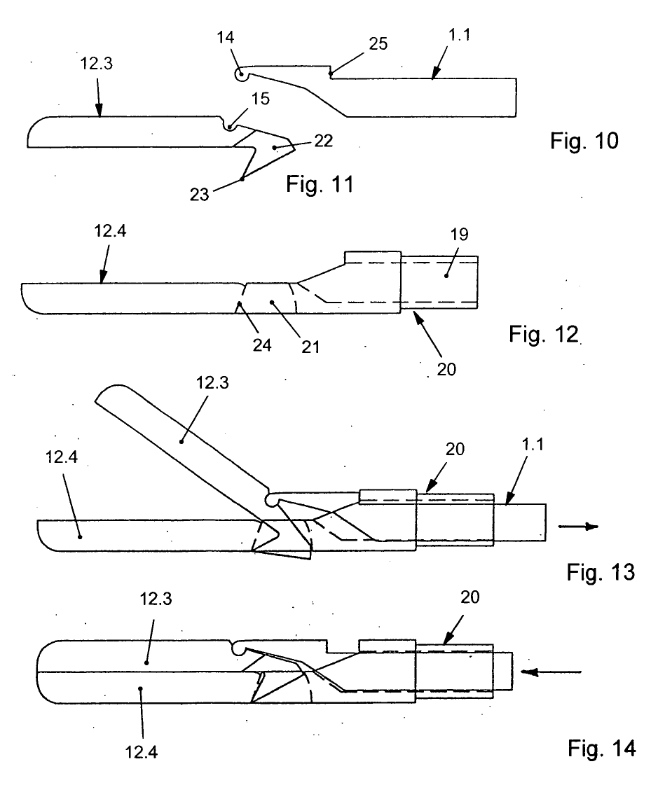

The arrangement of the bars 2.1 and 2.2 also permits a particular design of the movement mechanism forjaw parts 12.1 and 12.2, as is shown in FIGS. 6 through 9. Here, a front end 13 of the bar 2.1, 2.2 is made elastic and has a bead 14 at its free end. This bead 14 sits in a groove 15 formed in the jaw part 12.1, 12.2.

Each jaw part 12.1, 12.2 also has an attachment 16 with a bore 17 for receiving a hinge pin 18. This hinge pin 18 also extends through a bore (not shown) in the pulling or pushing element 1, this guaranteeing the movement of the jaw parts 12.1 and 12.2.

In the starting position shown in FIG. 6, the pulling or pushing element 1 is pushed forward onto the jaw parts 12.1 and 12.2, as is indicated by the corresponding arrow. The jaw parts 12.1 and 12.2 are thus opened, and the front ends 13 of the bars 2.1 and 2.2 are also spread outward.

If the pulling or pushing element 1 is now pulled back, as is indicated in FIG. 7 by the corresponding arrow, the hinge pin 18 and with it the jaw parts 12.1 and 12.2 are entrained toward the right, said jaw parts 12.1 and 12.2 turning with their grooves 15 about the beads 14. In this way, the jaw parts 12.1 and 12.2 close.

In the illustrative embodiment of the invention according to FIGS. 12 through 14, a surgical instrument is shown which opens on one side. A movable jaw part 12.3 again has a groove 15 for receiving the bead 14, which this time however is provided at a free end of a pulling or pushing element 1.1. This pulling or pushing element 1.1 passes through a recess 19 in an attachment piece 20, the latter comprising, after the recess 19, a further recess 21 which is adjoined by a rigid jaw part 12.4.

The movable jaw part 12.3 is inserted with a rotating end 22 into the recess 21, said rotating end 22 having an abutment tip 23 which in the use position bears against an inner wall 24 of the recess 21.

The respective arrows in FIGS. 13 and 14 indicate the pushing and pulling directions of the pulling or pushing element 1.1, the jaw being opened and closed by means of movement of the movable jaw part 12.3. The pulling or pushing element 1.1 slides in the recess 19, and a step 25 on the pulling or pushing element limits a movement of the element.

1. A surgical instrument with a pulling or pushing element for actuating at least one jaw part, wherein the pulling or pushing element is assigned at least one bar.

2. The surgical instrument as claimed in claim 1, wherein two bars are connected to one another.

3. The surgical instrument as claimed in claim 2, wherein the two bars are connected via at least one connecting bridge, a clamping ring, a guide rivet or the like.

4. The surgical instrument as claimed in claim 1, wherein, at least on one side, the pulling or pushing instrument is supported against a bar via at least one spacer.

5. The surgical instrument as claimed in claim 1, wherein the bars and the pulling or pushing element are located in a removable protective tube.

6. The surgical instrument as claimed in claim 5, wherein an outwardly directed outer contour of the bars is shaped to match an inner surface of the tube.

7. The surgical instrument as claimed in claim 5, wherein the protective tube is made of plastic.

8. The surgical instrument as claimed in claim 1, wherein the end of at least one bar is connected to a jaw part in a hinged manner.

9. The surgical instrument as claimed in claim 8, wherein the end of the bar has a bead which engages in a groove in the jaw part, said groove turning partially about the bead.

10. The surgical instrument as claimed in claim 8, wherein an end of the bar before the bead is designed to be elastic.

11. The surgical instrument as claimed in claim 8, wherein the jaw parts are connected to one another at their ends via a hinge pin.

12. The surgical instrument as claimed in claim 11, wherein the pulling or pushing element engages on the hinge pin.

13. A surgical instrument with a pulling or pushing element for actuating at least one jaw part, wherein the pulling or pushing element engages with a bead in a groove in the jaw part, and the jaw part engages with a rotating end into a recess in the other jaw part and rotates with the end in this recess.

14. The surgical instrument as claimed in claim 13, wherein the rotating end has an abutment tip which bears against an inner wall of the recess.
